(19) United States
(12) Patent Application Publication
(10) Pub. No.: US 2009/0171221 A1
(43) Pub. Date: Jul. 2, 2009

(54) System apparatus for monitoring heart and lung functions

(75) Inventors: Shu-Ting Liao, Taipei (TW); Chao-Fa Lee, Taipei (TW); Cheng-Hsing Kuo, Taipei (TW); Shih-Jung Chang, Taipei (TW)

Correspondence Address: BACON & THOMAS, PLLC, 625 SLATERS LANE, FOURTH FLOOR, ALEXANDRIA, VA, 22314-1176, US

(73) Assignee: Tatung Company, Taipei (TW)

(21) Appl. No.: 12/216,636

(22) Filed: Jul. 9, 2008

(30) Foreign Application Priority Data
Dec. 28, 2007 (TW) 096150795

(51) Int. Cl. A61B 5/0402; A61B 5/0205; A61B 7/00

(52) U.S. Cl. 600/484; 600/528; 600/529

(57) ABSTRACT

A system for monitoring heart and lung functions comprises an audio signal sensing unit, an audio signal processing unit, an electrocardiogram signal sensing unit, an electrocardiogram signal processing unit, and a microprocessor unit. The audio signal sensing unit senses audio signals, including a heart sound and a lung sound. The audio signal processing unit is connected to the audio signal sensing unit and processes the audio signals to obtain the heart sound and the lung sound. The electrocardiogram signal sensing unit senses an electrocardiogram signal. The electrocardiogram signal processing unit is connected to the electrocardiogram signal sensing unit and processes the electrocardiogram signal. The microprocessor unit is connected to the audio signal processing unit, the electrocardiogram signal processing unit and a computer host, and processes the heart sound, the lung sound and the electrocardiogram signal to be data that can be identified by the computer host.

### BACKGROUND OF THE INVENTION

1. Field of the Invention

The invention relates to a system and apparatus for monitoring heart and lung functions, particularly to a system and apparatus for monitoring heart and lung functions, capable of simultaneously measuring and analyzing a heart sound, a lung sound and an electrocardiogram signal.

2. Description of Related Art

In a general physiological monitoring system, various kinds of basic physiological parameters are used, in which a heart sound and a lung sound are important basis respectively for diagnosing heart and lung statuses. In diagnosing a heart disease, the status of the existing heart function and operation will be analyzed based on the relationship of relative position among electrocardiogram signal wave groups, incorporating with the appearance time of the heart sound.

The lung sound is a sound signal. The mechanism of sounding is that during air exchanging in the alveoli, the lung is proceeding with expanding and contracting, thereby causing movement of air fluid and then producing sound energy through vibration of air fluid in the breath tract. Except for an important basis for diagnosing a lung disease, monitoring the lung sound is even more important with respect to diseases possibly occurring together with rapid change of the lung.

During diagnosing the lung sound, accompanied noise quite often occurs. Hence, in processing and analyzing the signal of the lung sound, filtering of the environment noise is a very important step. Due to that the frequency spectrum of the environment noise is spread wide, a larger portion thereof will overlap with that of the lung sound. The source of the noise of the lung sound is divided into two kinds: noise in a body of a human being and nose outside the body. The noise in the body is essentially originated from the physiological signals resulting from activities of the internal organs, which signals are collected by the sensing element(s) during measuring, such as a heart sound. On the other hand, as the sensing element contacts the skin, contact noise will be produced due to activities of the skin and the muscle. The noise outside the body is essentially originated from the noise incurred in a meter itself. The environment noise refers to sound interference from the environment during measuring by a system, such as speaking sound and walking sound.

Traditionally, auscultation is used for distinguishing the heart sound and the lung sound. Therefore, a subjective factor, such as limitation of the inspector's sense of hearing, distinguishing ability or clinical experience, will possibly result in misjudging. Since the heart is a system dynamically changed depending on time, the heart noise occurring at this movement may disappear at once, resulting in difficulty of passing on experience.

Aiming at the above-mentioned problems, a digitized auscultation system of lung sound has been developed, but it is presented in a form of stethoscope, which needs to be operated by a doctor himself/herself. Such a system is not suitable for taking care in family or monitoring the lung status for a long time. In addition, the existing heart status recorder is mainly based on recording a single signal of the electrocardiogram signal or heart sound, while not providing the function of synchronously recording the electrocardiogram signal and heart sound, which is disadvantageous in diagnosing for locating heart diseases. If the heart sound and the lung sound cannot be analyzed synchronously, it will be difficult to locate the heart pathological changes to a particular position to analyze the function and status of heart operation.

Therefore, it is desirable to provide an improved system and apparatus for monitoring heart and lung functions to mitigate and/or obviate the aforementioned problems.

### BRIEF SUMMARY OF THE INVENTION

An objective of the invention is to provide a system for monitoring heart and lung functions, capable of concurrently obtaining a heart sound, a lung sound and an electrocardiogram signal and transferring the same to a computer host for analysis, while data obtained therefrom are important basis for disease diagnosing and health maintaining.

Another objective of the invention is to provide an apparatus for monitoring heart and lung functions, capable of concurrently measuring a heart sound, a lung sound and an electrocardiogram signal, so as to be served as important basis for disease diagnosing and health maintaining.

In accordance with one aspect of the invention, there is provided a system for monitoring heart and lung functions, comprising an audio signal sensing unit, an audio signal processing unit, an electrocardiogram signal sensing unit, an electrocardiogram signal processing unit, and a microprocessor unit. The audio signal sensing unit is used for sensing audio signals, including a heart sound and a lung sound. The audio signal processing unit is connected to the audio signal sensing unit and used for processing the audio signals to obtain the heart sound and the lung sound. The electrocardiogram signal sensing unit is used for sensing an electrocardiogram signal. The electrocardiogram signal processing unit is connected to the electrocardiogram signal sensing unit and used for processing the electrocardiogram signal. The microprocessor unit is connected to the audio signal processing unit, the electrocardiogram signal processing unit and a computer host, and used for processing the heart sound, the lung sound and the electrocardiogram signal to be data that can be verified by the computer host and are then transferred to the computer host.

According to the preferred embodiment of the system of the invention, the audio signal processing unit further comprises a first amplifier, connected to the audio signal sensing unit, for amplifying the audio signals.

According to the preferred embodiment of the system of the invention, the audio signal processing unit further comprises a low-pass filter, connected to the first amplifier, for eliminating noise signals in the audio signals.

According to the preferred embodiment of the system of the invention, the audio signal processing unit further comprises a high-pass filter, connected to the low-pass filter, for adjusting levels of the audio signals.

According to the preferred embodiment of the system of the invention, the electrocardiogram signal sensing unit further comprises a meter amplifier, connected to the electrocardiogram signal sensing unit, for obtaining the electrocardiogram signal.

According to the preferred embodiment of the system of the invention, the electrocardiogram signal processing unit further comprises a band-pass filter, connected to the meter amplifier, for eliminating noise signals in the electrocardiogram signal.

According to the preferred embodiment of the system of the invention, the electrocardiogram signal processing unit further comprises a second amplifier, connected to the band-pass filter, for amplifying the electrocardiogram signal.

In accordance with one aspect of the invention, there is provided an apparatus for monitoring heart and lung functions, including a case, an audio signal sensing unit, an audio signal processing unit, an electrocardiogram signal sensing unit, an electrocardiogram signal processing unit, and a microprocessor unit. The case has a surface. The audio signal sensing unit is installed on the surface of the case and used for sensing audio signals, including a heart sound and a lung sound. The audio signal processing unit is installed in the case and connected to the audio signal sensing unit, and is used for processing the audio signals to obtain the heart sound and the lung sound. The electrocardiogram signal sensing unit is used for sensing an electrocardiogram signal and provided with at least a first electrode and a second electrode, in which the first electrode is installed on the surface of the case and in the same location as the audio signal sensing unit. The electrocardiogram signal processing unit is connected to the electrocardiogram signal sensing unit and used for processing the electrocardiogram signal. The microprocessor unit is connected to the audio signal processing unit, the electrocardiogram signal processing unit and a computer host, and used for processing the heart sound, the lung sound and the electrocardiogram signal to be data that can be verified by the computer host and are then transferred to the computer host.

According to the preferred embodiment of the apparatus of the invention, the second electrode is installed at one end of the case.

According to the preferred embodiment of the apparatus of the invention, the electrocardiogram signal sensing unit further provides a third electrode.

According to the preferred embodiment of the apparatus of the invention, the third electrode is installed on the surface of the case and its location is different from the audio signal sensing unit.

### BRIEF DESCRIPTION OF THE DRAWINGS

FIG. 1 is a schematic diagram showing a system for monitoring heart and lung functions of a preferred embodiment of the invention; and

FIG.2 is a schematic diagram showing an apparatus for monitoring heart and lung functions of a preferred embodiment of the invention.

### DETAILED DESCRIPTION OF THE INVENTION

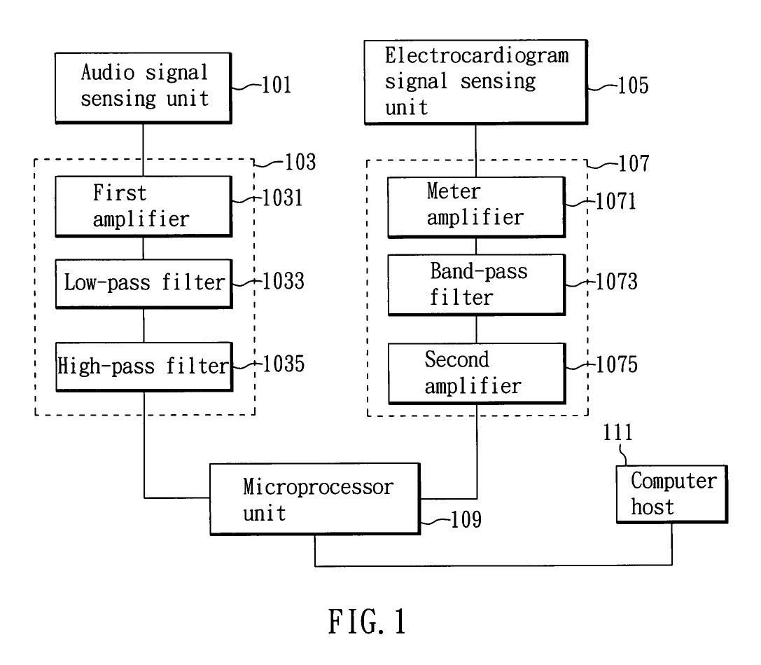

Please refer to FIG. 1. FIG. 1 is a schematic diagram showing a system for monitoring heart and lung functions of the preferred embodiment of the invention. The system comprises an audio signal sensing unit 101, an audio signal processing unit 103, an electrocardiogram signal sensing unit 105, an electrocardiogram signal processing unit 107, a microprocessor unit 109 and a computer host 111. The audio signal sensing unit 101 is used for sensing audio signals, including a heart sound and a lung sound. The audio signal processing unit 103 is connected to the audio signal sensing unit 101 and used for processing the audio signals to obtain the heart sound and the lung sound. The audio signal processing unit 103 comprises a first amplifier 1031, a low-pass filter 1033 and a high-pass filter 1035. The first amplifier 1031 is connected to the audio signal sensing unit 101 and used for amplifying the audio signals. The low-pass filter 1033 is connected to the first amplifier 1031 and used for eliminating noise other than the lung sound in the audio signals. The high-pass filter 1035 is connected to the low-pass filter 1033 and used for adjusting levels of the audio signals to avoid a large amount of distortion during analog-to-digital conversion.

The electrocardiogram (EKG) obtained from an electrocardiogram signal is an important method of recording electro-heart activities. In the embodiment, the electrocardiogram signal sensing unit 105 comprises a first electrode 1051, a second electrode 1052 and a third electrode 1053. The first electrode 1051 is located in a position capable of receiving the heart sound. The audio signal sensing unit 101 is, for example, a microphone, located on the first electrode 1051. The electrocardiogram signal processing unit 107 is connected to the electrocardiogram signal sensing unit 105 for processing an electrocardiogram signal and comprises a meter amplifier 1071, a band-pass filter 1073 and a second amplifier 1075. The meter amplifier 1071 is connected to the electrocardiogram signal sensing unit 107 for obtaining the electrocardiogram signal. The band-pass filter 1073 is connected to the meter amplifier 1071 for eliminating noise in the electrocardiogram signal. The second amplifier 1075 is connected to the band-pass filter 1073 for amplifying the electrocardiogram signal.

The microprocessor unit 109 is connected to the audio signal processing unit 103, the electrocardiogram signal processing unit 107 and the computer host 111, and used for processing the heart sound, the lung sound and the electrocardiogram signal to be data that can be verified by the computer host 111 and are then transferred to the computer host 111. The computer host 111 provides a storage device, such as a hard disk, for storing data regarding the heart sound, the lung sound and the electrocardiogram signal to be used for analysis in future. The way of transferring the data regarding the heart sound, the lung sound and the electrocardiogram signal from the microprocessor unit 109 to the computer host 111 may be in wired or wireless transmission. The microprocessor unit 109 may also be connected to a remote computer host 111 via a network for transferring the data regarding the heart sound, the lung sound and the electrocardiogram signal to the remote computer host via the network 111. Meanwhile, the computer host 111 may be a personal computer.

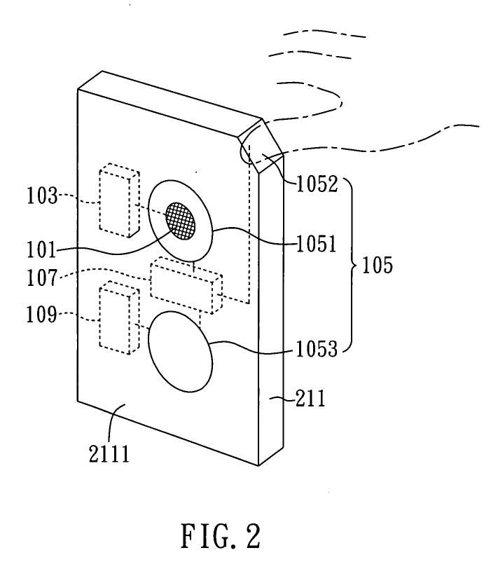

Please refer to FIG. 2. FIG. 2 is a schematic diagram showing an apparatus for monitoring heart and lung functions of a preferred embodiment of the invention. The apparatus for monitoring heart and lung functions comprises a case 211, an audio signal sensing unit 101, an audio signal processing unit 103, an electrocardiogram signal sensing unit 105, an electrocardiogram signal processing unit 107 and a microprocessor unit 109.

The case 211 has a surface 2111. The audio signal sensing unit 101 is installed on the surface 2111 of the case 211 and used for sensing audio signals, including a heart sound and a lung sound. The audio signal processing unit 103 is installed in the case 211 and connected to the audio signal sensing unit 101, and is used for processing the audio signals to obtain the heart sound and the lung sound. The electrocardiogram signal sensing unit 105 is used for sensing an electrocardiogram signal and provided with a first electrode 1051, a second electrode 1052 and a third electrode 1053. The electrocardiogram signal processing unit 107 is connected to the electrocardiogram signal sensing unit 105 and used for processing the electrocardiogram signal. The microprocessor unit 109 is connected to the audio signal processing unit 103, the electrocardiogram signal processing unit 107 and a computer host, and used for processing the heart sound, the lung sound and the electrocardiogram signal to be data that can be verified by the computer host and are then transferred to the computer host.

The first electrode 1051 in the electrocardiogram signal sensing unit 105 is installed on the surface of the case 211 and in the same location as the audio signal sensing unit 101. The audio signal sensing unit 101 may be such as a microphone. The second electrode 1052 is installed at one end of the case 211. In addition, the third electrode 1053 in the electrocardiogram signal sensing unit 105 serves as a co-electrode and is installed on the surface of the case 211 and not in the same location as the first electrode 1051.

According to the operation of the apparatus for monitoring heart and lung functions, the first electrode 1051 is placed near the heart such that the audio signal sensing unit 101 at the place of the first electrode 1051 is capable of receiving the heart sound and the lung sound. The place of the second electrode 1052 is pressed by a finger so that the first electrode 105 1, the second electrode 1052 and the third electrode 1053 are able to measure the correct electrocardiogram signals.

Further, the second electrode 1052 and the third electrode 1053 may be installed on an external place extending from the case 211. For example, the second electrode 1052 is placed on the left hand and the third electrode 1053 is placed on the left foot.

The apparatus for monitoring heart and lung functions of the invention may be in the form of such as a personal digital assistant (PDA). Through a wireless web-card, high speed operation ability provided by the PDA and large capacity of memory, the data stored in the database of the PDA may be transferred to a remote server including patient database for analyzing and recording conveniently by medical personnel, while the health of participant can be monitored and understood by himself any time through the PDA.

The system for monitoring heart and lung functions of the invention can simultaneously obtain the heart and lung sounds and the electrocardiogram signal to facilitate analyzing and verifying human's manifestations of a disease and to store the measuring result based for future reference.

Although the present invention has been explained in relation to its preferred embodiment, it is to be understood that many other possible modifications and variations can be made without departing from the spirit and scope of the invention as hereinafter claimed.

What is claimed is:

1. A system for monitoring heart and lung functions, comprising:
an audio signal sensing unit for sensing audio signals, including a heart sound and a lung sound;
an audio signal processing unit, connected to the audio signal sensing unit, for processing the audio signals to obtain the heart sound and the lung sound;
an electrocardiogram signal sensing unit for sensing an electrocardiogram signal;
an electrocardiogram signal processing unit, connected to the electrocardiogram signal sensing unit, for processing the electrocardiogram signal; and
a microprocessor unit, connected to the audio signal processing unit, the electrocardiogram signal processing unit and a computer host, for processing the heart sound, the lung sound and the electrocardiogram signal to be data that can be verified by the computer host and are then transferred to the computer host.

2. The system as claimed in claim 1, wherein the audio signal processing unit further comprises a first amplifier, connected to the audio signal sensing unit, for amplifying the audio signals.

3. The system as claimed in claim 2, wherein the audio signal processing unit further comprises a low-pass filter, connected to the first amplifier, for eliminating noise signals in the audio signals.

4. The system as claimed in claim 3, wherein the audio signal processing unit further comprises a high-pass filter, connected to the low-pass filter, for adjusting levels of the audio signals

5. The system as claimed in claim 1, wherein the electrocardiogram signal sensing unit comprises a first electrode, a second electrode and a third electrode.

6. The system as claimed in claim 5, wherein the first electrode is placed in a position capable of receiving the heart sound.

7. The system as claimed in claim 6, wherein the audio signal sensing unit is a microphone located on the first electrode.

8. The system as claimed in claim 1, wherein the electrocardiogram signal processing unit further comprises a meter amplifier connected to the electrocardiogram signal sensing unit for obtaining the electrocardiogram signal.

9. The system as claimed in claim 8, wherein the electrocardiogram signal processing unit further comprises a band-pass filter connected to the meter amplifier for eliminating noise signals in the electrocardiogram signal.

10. The system as claimed in claim 9, wherein the electrocardiogram signal processing unit further comprises a second amplifier connected to the band-pass filter for amplifying the electrocardiogram signal.

11. The system as claimed in claim 1, wherein the microprocessor unit transfers the heart sound, the lung sound and the electrocardiogram signal to the computer host in wireless transmission.

12. The system as claimed in claim 1, wherein the computer host provides a storage device for storing data regarding the heart sound, the lung sound and the electrocardiogram signal.

13. The system as claimed in claim 1, wherein the microprocessor unit is connected to a remote computer host via a network for transferring data regarding the heart sound, the lung sound and the electrocardiogram signal to the remote computer host via the network.

14. The system as claimed in claim 1, wherein the computer host is a personal computer.

15. An apparatus for monitoring heart and lung functions, comprising:
a case with a surface;
an audio signal sensing unit, installed on the surface of the case, for sensing audio signals, including a heart sound and a lung sound;
an audio signal processing unit, installed in the case and connected to the audio signal sensing unit, for processing the audio signals to obtain the heart sound and the lung sound;
an electrocardiogram signal sensing unit for sensing an electrocardiogram signal, provided with at least a first electrode and a second electrode, the first electrode installed on the surface of the case and in the same location as the audio signal sensing unit;
an electrocardiogram signal processing unit, connected to the electrocardiogram signal sensing unit, for processing the electrocardiogram signal; and
a microprocessor unit, connected to the audio signal processing unit, the electrocardiogram signal processing unit and a computer host, for processing the heart sound, the lung sound and the electrocardiogram signal to be data that can be verified by the computer host and are then transferred to the computer host.

16. The apparatus as claimed in claim 15, wherein the second electrode is installed at one end of the case.

17. The apparatus as claimed in claim 16, wherein the second electrode is installed at an external place extended from the case.

18. The apparatus as claimed in claim 15, wherein the electrocardiogram signal sensing unit further provides a third electrode.

19. The apparatus as claimed in claim 18, wherein the third electrode is installed on the surface of the case and in a different location from the audio signal sensing unit.

20. The apparatus as claimed in claim 18, wherein the third electrode is installed at an external place extended from the case.
